(19) United States
(12) Patent Application Publication
(10) Pub. No.: US 2022/0105223 A1
(43) Pub. Date: Apr. 7, 2022

(54) AIR FRESHENER DESIGN FOR OPTIMIZED AIR FLOW

(71) Applicant: HENKEL IP & HOLDING GmbH, Duesseldorf (DE)

(72) Inventors: Terannie Vazquez Alvarez, Orange, CT (US); Mark A. Granja, Danbury, CT (US); Glenn William Kaye, Norwalk, CT (US); Edward Gilchrest, Oxford, CT (US); Andrew Bell Krystinik, Middlefield, CT (US); Elana Rae Abrams, Shelton, CT (US)

(73) Assignee: HENKEL IP & HOLDING GMBH, Duesseldorf (DE)

(21) Appl. No.: 17/491,325

(22) Filed: Sep. 30, 2021

Related U.S. Application Data
(60) Provisional application No. 63/198,231, filed on Oct. 5, 2020.

(51) Int. Cl. A61L 9/12

(52) U.S. Cl. CPC A61L 9/122; A61L 2209/133; A61L 2209/134

(57) ABSTRACT

A volatile substance distribution system includes a fan having a fan profile. The system includes a housing that defines a fluid passage. The fan is disposed in the fluid passage. The housing defines a receptacle configured to removably receive a capsule containing a volatile substance member. The fluid passage is fluidly connected to the capsule when the receptacle removably receives the capsule. The system further includes a shroud surface that partly defines the fluid passage and that opposes the fan. The shroud surface and the fan profile have a corresponding contour.

### RELATED APPLICATION

The following claims priority to U.S. Provisional Patent Application 63/198,231, filed Oct. 5, 2020, the entire disclosure of which is incorporated by reference.

### TECHNICAL FIELD

The following relates to a volatile substance distribution system and, more particularly, relates to a volatile substance distribution system with an airflow system having a shrouded fan.

### BACKGROUND

There are various devices used to distribute volatile materials (e.g., perfumes, essential oils, insect repellant, etc.) into the air. Many devices include a unit that supports the volatile material and a fan. The volatile material moves into the airstream moved by the fan for distribution into the air.

However, conventional systems suffer from various drawbacks. For example, many of these devices are bulky. Also, the fan may consume relatively high levels of electricity during use. These and other concerns may considerably limit the usefulness of conventional systems. As an example, a conventional system may not be suitable for use in a vehicle because the system may be too big, may require a power cord, etc.

Therefore, there exists a need for a compact and efficient volatile material distribution system. Other desirable features and characteristics of the devices and methods of the present disclosure will become apparent from the subsequent detailed description and the appended claims, taken in conjunction with the accompanying drawings and the preceding background.

### BRIEF SUMMARY

Embodiments of a volatile substance distribution system are provided. In various embodiments, the system includes a fan having a fan profile. The system includes a housing that defines a fluid passage. The fan is disposed in the fluid passage. The housing defines a receptacle configured to removably receive a capsule containing a volatile substance member. The fluid passage is fluidly connected to the capsule when the receptacle removably receives the capsule. The system further includes a shroud surface that partly defines the fluid passage and that opposes the fan. The shroud surface and the fan profile have a corresponding contour.

Embodiments of a method of manufacturing a volatile substance distribution system are also provided. In some embodiments, the method includes supporting a fan within a fluid passage of a housing. The fan has a fan profile. The housing defines a receptacle configured to removably receive a capsule containing a volatile substance member. The fluid passage fluidly connects to the capsule when the receptacle removably receives the capsule. The method also includes supporting the fan within a shroud surface that partly defines the fluid passage and that opposes the fan. The shroud surface and the fan profile have a corresponding contour.

Furthermore, embodiments of a volatile substance distribution system are provided. In some embodiments, the volatile substance distribution system includes a capsule containing a volatile substance member. The system also includes a base unit with a housing that houses a fan having a fan profile. The housing defines a receptacle that removably receives the capsule. The housing defines a fluid passage that fluidly connects to the capsule. The fan is disposed in the fluid passage. The fluid passage is partly defined by a shroud surface of the housing. The shroud surface opposes the fan. The shroud surface and the fan profile have a corresponding contour.

The foregoing statements are provided by way of non-limiting example only. Various additional examples, aspects, and other features of embodiments of the present disclosure are encompassed by the present disclosure and described in more detail below.

### BRIEF DESCRIPTION OF THE DRAWINGS

At least one example of the present disclosure will hereinafter be described in conjunction with the following figures, wherein like numerals denote like elements, and:

FIG. 1 is a perspective view of a volatile substance distribution system according to example embodiments of the present disclosure;

FIG. 2 is a perspective view of a base unit of the system of FIG. 1;

FIG. 3 is a perspective view of a capsule of the system of FIG. 1;

FIG. 4 is an isometric section view of the base unit and the capsule of the system of FIG. 1;

FIG. 5 is an axial section view of the base unit and the capsule of the system of FIG. 1; and

FIG. 6 is a lateral section view of the base unit of the system of FIG. 1.

For simplicity and clarity of illustration, descriptions and details of well-known features and techniques may be omitted to avoid unnecessarily obscuring the exemplary and non-limiting embodiments of the present disclosure described in the subsequent Detailed Description. It should further be understood that features or elements appearing in the accompanying figures are not necessarily drawn to scale unless otherwise stated.

### DETAILED DESCRIPTION

The following Detailed Description is merely exemplary in nature and is not intended to limit the present disclosure or the application and uses of the same. The term “exemplary,” as appearing throughout this document, is synonymous with the term “example” and is utilized repeatedly below to emphasize that the following description provides only multiple non-limiting examples of the present disclosure and should not be construed to restrict the scope of the present disclosure, as set-out in the Claims, in any respect.

Systems for distributing a volatile substance are provided, as are methods for operating and manufacturing such systems. Generally, the systems described herein may include a base unit and a capsule that may be removably engaged with the base unit. The capsule may contain a volatile substance member and may receive an airflow that is driven by a fan of the base unit. As the airflow moves through the capsule, the volatile substance may enter the airstream for distribution outside the system.

The base unit and capsule may cooperatively define an airflow system. The capsule and/or base unit may include various features that improve efficiency such that the fan effectively drives air through the system. In addition, the base unit and capsule may be very compact.

In some embodiments, for example, the fan of the airflow system may be shrouded. A shroud member may surround the fan and these components may have corresponding contours, shapes, profiled, etc. There may be minimal clearance defined between the ends of the fan blades and an inner surface of the shroud. Additionally, the shroud may be disposed within and may define a portion of a flow channel of the airflow system that extends through the base unit and the capsule. The inner surface of the shroud may include one or more tapered surfaces or other features for efficiently directing and funneling fluid through the flow channel.

Furthermore, in some embodiments, the base unit may include a chassis that supports the fan and one or more batteries for powering the fan. The chassis may further define one or more apertures that are fluidly connected to the shroud of the fan. The chassis provides robust support for the fan and batter(ies) and, yet, the base unit may still be very compact. The chassis may also improve efficiency of the system.

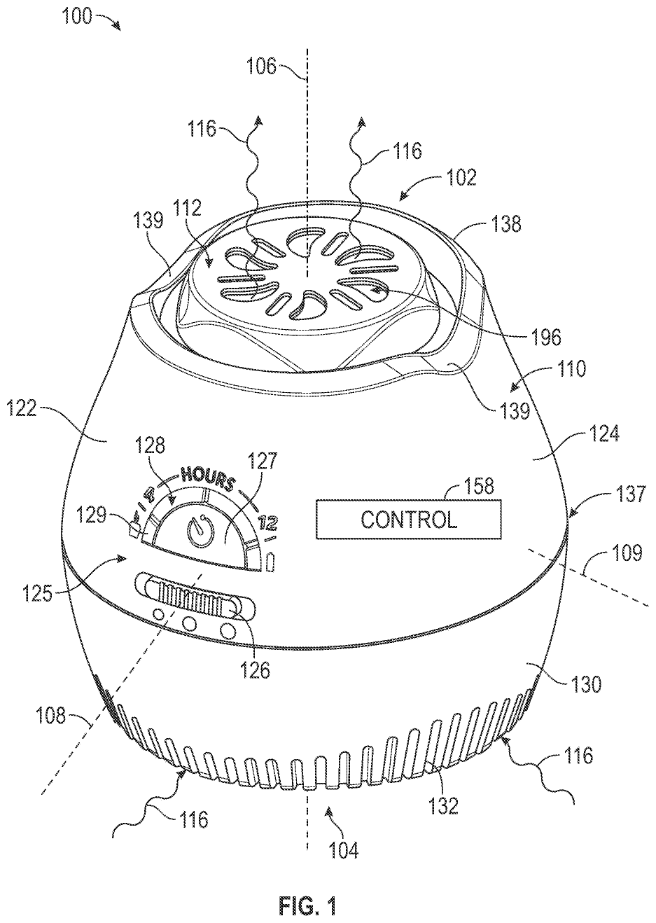

A volatile substance distribution system 100 will now be discussed according to example embodiments illustrated in FIG. 1. Generally, the system 100 includes an upper end 102 and a lower end 104 and a longitudinal axis 106 that extends therebetween. It will be appreciated that the terms “upper” and “lower” are relative terms based on the orientation shown in the Figures and are merely used as an example. Accordingly, the upper end 102 may be referred to as a “first end” and the lower end 104 may be referred to as a “second end.” A first radial axis 108 and a second radial axis 109, which are normal to each other, are also indicated in relation to the longitudinal axis 106 for reference purposes.

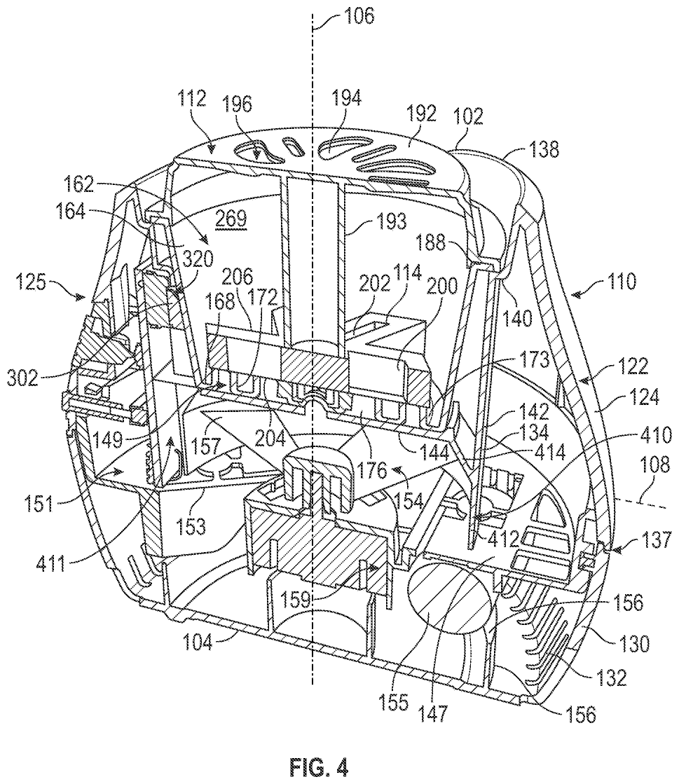

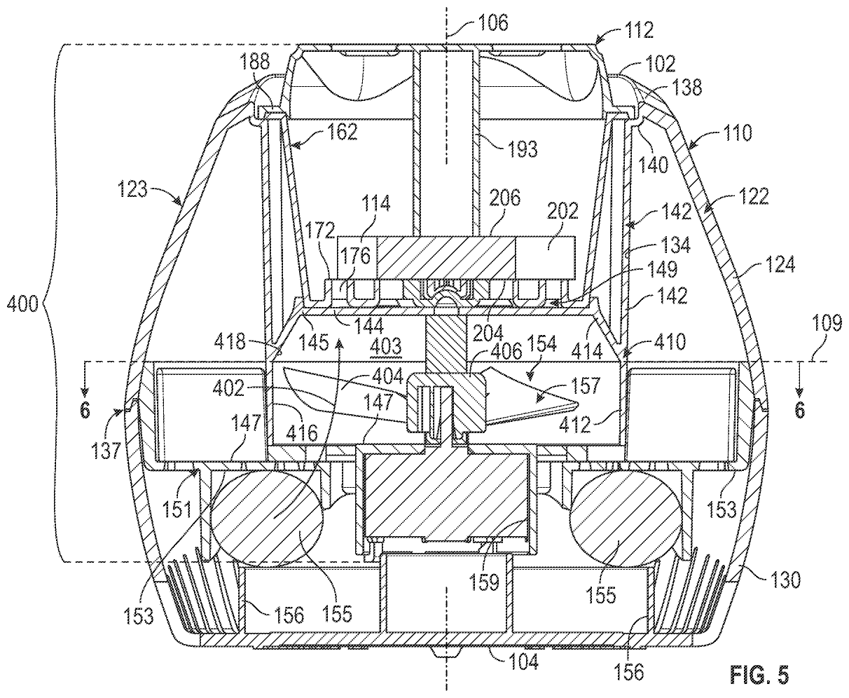

The volatile substance distribution system 100 may include a base unit 110 (FIGS. 1, 2, 4, and 5) and at least one volatile substance capsule 112 (FIGS. 1 and 3-5). In the illustrated embodiments, the base unit 110 may be configured for supporting a single capsule 112; however, in other embodiments, the base unit 110 may be configured for supporting multiple capsules 112. In some embodiments, the capsule 112 is a replaceable unit that may be removably supported by the base unit 110. The capsule 112 may also be referred to as a refill unit, as a cup or other container, as a pod, or as another term. The capsule 112 may be a single-use, disposable container, or the capsule 112 may be a refillable/reusable container. The capsule 112 may also be recyclable in some embodiments. The system 100 may additionally include a volatile substance member 114 that is contained within the capsule 112 (FIGS. 4 and 5). The volatile substance member 114 may include, contain, or otherwise comprise a volatile substance, such as an air freshener, essential oil, perfume, aromatherapy or homeopathy substances, materials for malodor control, insect control substances, etc. The term “volatile substance” as used herein will be understood broadly to include substances that readily vaporize and/or move into the air. In some embodiments, the system 100 may be configured for volatile substances that vaporize and move into an airstream flowing through the capsule 112 at normal ambient temperatures. As represented in FIG. 1 and as will be described in detail, the system 100 may operate with the base unit 110 driving airflow (represented by arrow 116) through the capsule 112. The airflow 116, therefore, may carry the volatile substance from the member 114 and distribute it throughout the air outside the capsule 112. However, it will be appreciated that the system 100 may be otherwise configured, for example, to include a heating system for heating and vaporizing volatile substances, a wick, and/or other elements for delivering volatile substances into the air.

Referring now to FIGS. 1, 2, and 4, the base unit 110 will be discussed in detail according to example embodiments. The base unit 110 may include a housing 122. The housing 122 may be a relatively thin-walled or shell-like rigid structure constructed from one or more pieces. The piece(s) of the housing 122 may define an outer side member 124, a bottom member 130, and an inner member 134.

The outer side member 124 may be frusto-conic in shape. The outer side member 124 may be substantially centered about the longitudinal axis 106. The outer side member 124 may taper outward in width as the outer side member 124 extends from the upper end 102 toward the lower end 104. The outer side member 124 may have an arcuate or rounded (e.g., circular, ovate, etc.) cross section taken perpendicular to the axis 106. The outer side member 124 may support a user interface 125, which may include one or more user input devices and/or one or more user output devices.

The bottom member 130 of the housing 122 may be rounded and bowl-shaped. The bottom member 130 may be fixedly attached to the lower rim of the outer side member 124 of the housing 122 and may define the lower end 104. The bottom member 130 may include a relatively flat or otherwise supportive bottom surface for standing the bottom base unit 110 upright. The bottom member 130 may have a rounded cross section taken perpendicular to the longitudinal axis 106. In some embodiments, the width of the bottom member 130 (measured perpendicular to the axis 106) and the shape of the bottom member 130 may be configured for certain uses and environments. For example, the bottom member 130 may be sized and shaped to fit within a standard vehicle cupholder. Thus, the rounded shape and relatively small width may allow the base unit 110 to be securely received in the cup holder and the system 100 can freshen air within a vehicle.

The bottom member 130 may also include a plurality of apertures 132 (first apertures or inlet apertures). The apertures 132 may be elongate slots that extend through the thickness of the bottom member 130. In some embodiments, the apertures 132 may provide an inlet passage for the airflow 116 into the base unit 110.

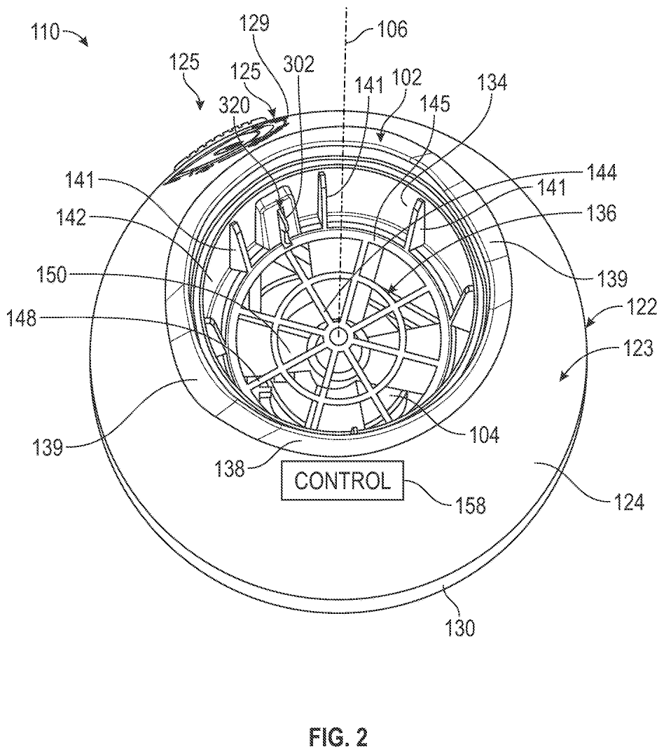

As shown in FIGS. 2, 4, and 5, the inner member 134 of the housing 122 may be cup-shaped and may be attached to the outer side member 124 along an upper rim 138 of the end 102. The inner member 134 may be integrally attached to the outer side member 124 at the upper rim 138 so as to define a unitary, one-piece upper member 123. This upper member 123 may be thin-walled and shell-like. The annular lower rim of the upper member 123 may be removably attached to the bottom member 130 at a circumferentially-extending housing junction 137. The junction 137 may removably attach the upper member 123 and the bottom member 130, and the junction 137 may include interlocking retainer features that may be manually attached and detached.

The cup-shaped inner member 134 may define a receptacle 136 of the housing 122. The receptacle 136 may be open at the upper end 102. The receptacle 136 may extend from the upper rim 138 and may be recessed therefrom, toward the lower end 104 along the axis 106. The receptacle 136 may be centered about the axis 106. The receptacle 136 may be shaped and sized according to the capsule 112. Thus, in some embodiments, the receptacle 136 may define a rounded cup-like recess for receiving the slightly-smaller capsule 112. The depth of the receptacle 136 may be sufficient to receive the majority of the capsule 112. For example, as shown in FIGS. 1, 4, and 5, the receptacle 136 may be deep enough such that capsule 112 is nested with the upper rim and topside of the capsule 112 remaining exposed. The receptacle 136 may also be referred to as a docking station for the capsule 112.

In some embodiments, the upper rim 138 may include at least one notch 139. As shown in FIGS. 1 and 2, there may be two notches 139 that are spaced apart on opposite sides of the axis 106. The upper rim 138 may be scalloped with gradual contours to define the notches 139. The notches 139 may provide access to the capsule 112 for grasping and removing the capsule 112 from the base unit 110.

The inner member 134 of the housing 122 may include an inner ledge 140 (FIGS. 4 and 5) that is disposed downward axially from the upper rim 138. The inner ledge 140 may extend substantially perpendicular to the axis 106 and inward radially toward the axis 106. The inner ledge 140 may be annular and may extend about the axis 106. The inner member 134 and the receptacle 136 may include a side wall 142, which may be substantially cylindrical, and which may depend downward along the axis 106 from the ledge 140. As shown in FIG. 2, the inner member 134 may include a plurality of elongate ribs 141 that extend longitudinally along the side wall 142 and that project slightly inward radially toward the axis 106. The ribs 141 may be spaced apart substantially equally in the circumferential direction about the axis 106. Additionally, the inner member 134 and the receptacle 136 may include a lower support 144 (FIG. 2). The lower support 144 may be a frusto-conic platform that is attached to the side wall 142 and that extends inwardly therefrom. The lower support 144 may include an outer ledge 145 and a plurality of elongate support members 148 that extend radially inward from the outer ledge 145 and that are connected at a central hub, for example, in a web-shaped arrangement.

The base unit 110 may further include an air outlet 150 that is defined between the elongate support members 148. Thus, the air outlet 150 may extend through the lower support 144. The air outlet 150 may be in fluid communication with the interior of the housing 122 and with the apertures 132 of the bottom member 130. As such, the airflow 116 may move through the base unit 110 from the apertures 132 (the inlet), through the housing 122, and out of the housing 122 via the air outlet 150. As will be discussed, the air outlet 150 may blow air out of the base unit 110, upward along the axis 106, and into the capsule 112 in a downstream flow direction through the capsule 112.

The inner member 134 of the housing 122 may additionally include an abutment member 302 that is supported for movement between a neutral position (FIG. 2) and an actuated position (FIG. 4). When positioned in the receptacle 136, the capsule 112 may abut against the abutment member 302 and hold it in the actuated position for detecting that the capsule 112 is seated and/or engaged with the base unit 110 and is ready for use.

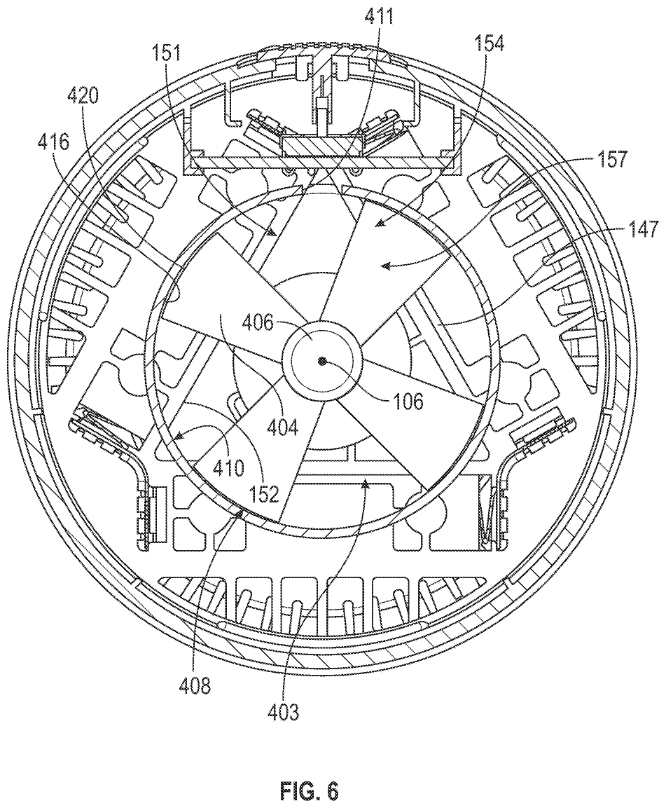

The base unit 110 may additionally include an internal chassis 151 (FIGS. 4-6). The chassis 151 may be rounded (e.g., circular), and the chassis 151 may be substantially flat and plate-like. The chassis 151 may be constructed of a rigid material, such as a polymeric material, and the chassis 151 may be constructed of the same material as the other members of the housing 122 in some embodiments. The chassis 151 may be disposed substantially perpendicular to the axis 106 and may extend laterally across the interior of the housing 122. The chassis 151 may be attached at its periphery to the outer side member 124 and/or to the bottom member 130. The chassis 151 may include one or more apertures 152 (FIG. 6). As shown, there may be a central aperture 152 through the chassis 151, and the aperture 152 may be substantially centered on the axis 106. The aperture 152 may be substantially triangular in some embodiments as represented in FIG. 6. The aperture 152 may have a profile resembling a triangle that is centered on the axis 106. The aperture 152 may allow the airflow 116 to pass through the chassis 151 as it passes from the lower end 104 toward the capsule 112.

The chassis 151 may include an underside 153 that supports one or more batteries 155. The underside 153 may include retaining members, electrical terminals, and/or other features for arranging the batteries 155 in a compact manner. For example, in some embodiments, there may be three batteries 155, which are arranged end-to-end in an equilateral triangular formation that is centered about the axis 106. This arrangement may evenly distribute weight of the batteries 155 to provide stability to the system 100 and prevent tipping. In this formation, the batteries 155 may leave a considerable area of the aperture 152 open and exposed for airflow therethrough.

As shown in FIGS. 4 and 5, the interior side of the bottom member 130 may include projecting structures 156, such as walls, fins, posts, or other structures that project upwardly. The structures 156 may be annular in some embodiments. The structures 156 may help support the batteries 155 and hold the batteries 155 to the chassis 151 in some embodiments. The underside 153 of the chassis 151 may also include a central cavity 159.

The base unit 110 may further include a fan 154. The fan 154 may be an electrical fan with a motor supported within the central cavity 159. As such, the motor of the fan 154 may be supported on the underside 153 of the chassis 151. The fan 154 may be compact and may have relatively low power requirements so that it can be battery-powered. The fan 154 may include a rotor 157 that extends through the aperture 152 of the chassis 151. The rotor 157 may include a plurality of blades supported above a topside 147 of the chassis 151. The rotor 157 may be supported for rotation about the axis 106 such that the blades of the rotor 157 drive the airflow 116 through the housing 122 and toward the capsule 112 via the air outlet 150. More specifically, the rotor 157 may be supported for rotation about the axis 106 to draw the airflow 116 radially into the base unit 110 via the apertures 132 in the bottom member 130, through the aperture 152 in the chassis 151, and out the base unit 110 via the air outlet 150, generally along the axis 106.

It will be appreciated that the system 100 may be configured differently for moving air through the capsule 112. For example, instead of or in addition to the fan 154 the system 100 may incorporate an air pump, moveable bellows, air multipliers, or other features. Additionally, the fan 154 may be positioned differently from the illustrated embodiments without departing from the scope of the present disclosure. Moreover, as represented by the illustrated embodiment, the fan 154 may be configured for positive displacement relative to the capsule 112 such that the fan 154 drives (blows) the airflow 116 into the capsule 112. However, it will be appreciated that the fan 154 of the system 100 may be configured for negative displacement relative to the capsule 112 such that the fan 154 drives (sucks) air through the capsule 112. Moreover, instead of or in addition to the fan 154, the system 100 may include other features for moving volatiles out of the capsule 112, such as a heating element, etc. Furthermore, the system 100 may be configured for delivering volatiles passively and without relying on a power source to input power.

As mentioned above, the base unit 110 may include a user interface 125. The user interface 125 may have a variety of configurations without departing from the scope of the present disclosure. For example, as shown in FIG. 1, the user interface 125 may include one or more input devices 126, 127 and at least one output device 128.

In some embodiments, a first input device 127 may be a button. In some embodiments, the first input device 127 may be pressed once to turn ON the fan 154 and keep the fan 154 rotating continuously for a predetermined time interval (e.g., continuously for four hours) before being automatically shut OFF. Additionally, the first input device 127 may be pressed a second time to turn ON the fan 154 and keep the fan 154 rotating continuously for a second predetermined time interval (e.g., continuously for twelve hours). Furthermore, the first input device 127 may be pressed a third time to manually turn OFF the fan 154.

Furthermore, in some embodiments, a second input device 126 may be a sliding switch that may be actuated for changing dispersion intensity of the volatile materials from the system 100. In some embodiments, the second input device 126 may be actuated for changing the speed of the fan between various speed settings, thereby changing dispersion intensity by the system 100.

Also, the output device 128 may include at least one visual output device 129 (FIG. 1). The visual output device 129 may include one or more lamps, LEDs, etc. There may be a plurality of different output devices 129 for indicating different information about the system 100. Also, in some embodiments a single output device 129 may provide a plurality of different signals that indicate different information about the system 100. It will be appreciated that the output device 128 may include an audio output device or other output device without departing from the scope of the present disclosure. Accordingly, the output device 128 may indicate that the fan 154 is ON. The output device 128 may also be configured for indicating whether power levels are low (e.g., to indicate that batteries should be changed). Furthermore, as will be discussed, the output device 128 may be configured for indicating when to change the capsule 112.

The base unit 110 may house a control system 158 within the housing 122. The control system 158 may be of a variety of types and may have a wide range of capabilities without departing from the scope of the present disclosure. In some embodiments, the control system 158 may include a processor, a memory device, sensor(s), and/or other components of a known computerized control system. Furthermore, the control system 158 may rely on programmed logic, sensor input, and/or stored data for controlling one or more features of the system 100.

For example, the control system 158 may be operably connected to the fan 154 for turning the fan 154 ON and OFF. In some embodiments, the control system 158 may be operably attached to the input device 127 to turn the fan 154 ON and OFF according to the user's input. In some embodiments, the user may input a first command (e.g., a first push of the input device 127), and the control system 158 may, in turn, continuously run the fan 154 for a first time interval (e.g., for four hours) before automatically shutting OFF the fan 154. Additionally, the user may input a second command (e.g., a second push of the input device 127), and the control system 158 may, in turn, continuously run the fan 154 for a second time interval (e.g., for twelve hours) before automatically shutting OFF the fan 154. The user may input a third command (e.g., a third push of the input device 127) to manually shut OFF the fan 154. The control system 158 may also adjust the speed of the fan 154 between two or more predetermined speed settings (e.g., Low speed, Medium speed, and High speed) based on the position of the second input device 126.

Referring now to FIGS. 1 and 3-5, the capsule 112 will be discussed in detail according to example embodiments. The capsule 112 may include a housing 162, which houses the volatile substance member 114 (FIG. 4). The housing 162 may be hollow and cup-shaped. In some embodiments, the housing 162 may be substantially cylindrical and may have a generally circular cross section taken normal to the axis 106. The housing 162 may be centered on the axis 106 and may extend along the axis 106 between a first end 161 (i.e., a bottom or inlet end) and a second end 163 (i.e., a top or outlet end). The first end 161 may be oriented toward the lower end 104 and the second end 163 may be disposed proximate the upper end 102 when mounted on the base unit 110.

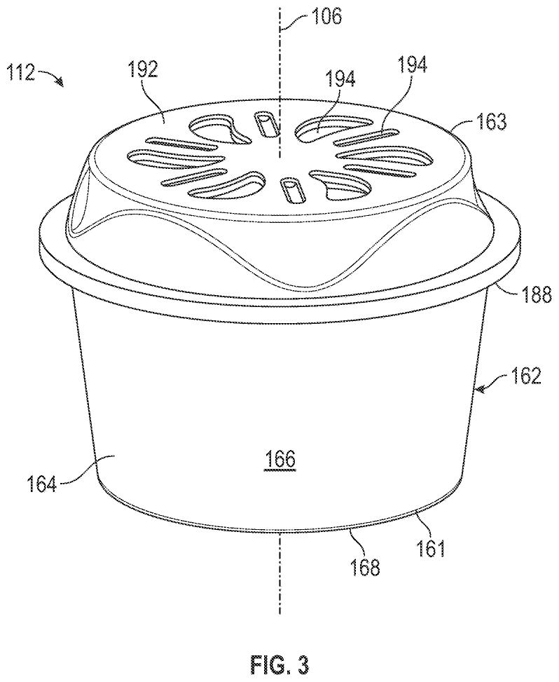

As shown in FIG. 3, the housing 162 may generally include a cup member 164 and a cover member. The cup member 164 and cover member 192 may cooperate to house, encapsulate, and/or retain the volatile substance member 114 therein.

The cup member 164 may be a unitary member made of a polymeric material. The cup member 164 may be somewhat flexible but may be rigid enough to support itself and contents therein. The cup member 164 may include an outer wall 166 that extends circumferentially about the longitudinal axis 106. The outer wall 166 may be centered on the axis 106. The outer wall 166 may also extend along the longitudinal axis 106 in a first direction (downward) toward the first end 161 and may terminate at a first terminal end 168 of the capsule 112. The outer wall 166 may also include an upper rim 188, which is spaced apart longitudinally from the first terminal end 168 of the capsule 112. The outer wall 166 may have a circular cross section taken normal to the axis 106. In other embodiments, the outer wall 166 may have a different shape, such as a square or other polygonal shape. The outer wall 166 may be frusto-conic and tapered slightly with respect to the axis 106. As such, the outer wall 166 proximate the first end 161 may be narrower than the outer wall 166 proximate the second end 163.

The cup member 164 may include a lower platform 172, which is disposed proximate the first terminal end 168. The lower platform 172 may span across the first terminal end 168 and may be attached at its periphery to the outer wall 166. The lower platform 172 may be offset in the longitudinal direction from the first terminal end 168 so as to define an annular trough 173 at the periphery of the lower platform 172 and proximate the outer wall 166. The lower platform 172 may define an air inlet 176 (e.g., at least one opening) extending therethrough in the axial direction. The lower platform 172 may support the volatile substance member 114 thereon such that air passing through the air inlet 176 flows over and past the volatile substance member 114.

The cover member 192 may be a frusto-conic disc that is attached at its periphery to the upper rim 188 of the cup member 164. The cover member 192 may be made of a polymeric material. In some embodiments, the cover member 192 may be welded (i.e., plastic welded) to the cup member 164, although it will be appreciated that the cover member 192 may be adhesively attached or otherwise fastened to the cup member 164 without departing from the scope of the present disclosure. The cover member 192 may include a plurality of apertures 194. The apertures 194 may have a variety of shapes without departing from the scope of the present disclosure, such as slot-shaped apertures 194, teardrop shaped apertures 194, or other shapes. As will be discussed, the apertures 194 may define an outlet port 196 for the capsule 112.

A part of the volatile substance member 114 is shown in FIG. 4 according to example embodiments. The volatile substance member 114 in some embodiments may include a substrate 200 with a volatile substance absorbed thereon. For example, the substrate 200 may be made from a sheet of material (e.g., cotton, paper, plant-based material, non-woven material, porous or spiralized plastic, polymeric material, corrugated sheet, sponge material, etc.) with fragrance oil thereon. Accordingly, the volatile substance member 114 may be substantially dry and moisture-free inside the capsule 112 during normal consumer use to avoid any spillage or leakage of fragrance oil. In other embodiments, the volatile substance member 114 and/or the substrate 200 may comprise beads, particles, etc. that are scented with a fragrance oil. In further embodiments, the volatile substance member 114 may include a container for a fragrant gel, fragrance oil, a wick, or other features without departing from the scope of the present disclosure.

The substrate 200 may be formed in a variety of shapes without departing from the scope of the present disclosure. For example, the substrate 200 may be arranged substantially in a star-shape that is centered on the axis 106. The substrate 200 may include a first side 204 and a second side 206. The first side 204 may face the lower end 104, and the second side 206 may face the upper end 102. A plurality of through-ways 202 may be defined through the volatile substance member 114 along the axis 106 from the first side 204 to the second side 206. The volatile substance member 114 may also be heart-shaped, rectangular, triangular, or shaped otherwise.

The volatile substance member 114 may be supported atop the lower platform 172 of the cup member 164 and may be centered thereon. Also, the cover member 192 may include a projecting member 193 (FIGS. 4 and 5) that projects and depends from the inside top surface to abut against the second side 206 of the volatile substance member 114. Accordingly, the lower platform 172 and the projecting member 193 may cooperate to retain the volatile substance member 114 in place.

The first side 204 and the second side 206 may be open such that air passing through the capsule 112 may pass over and through the volatile substance member 114. Accordingly, there may be a relatively high amount of exposed surface area for passing the volatile substance to the airflow 116.

To use the system 100, packaging may be removed from the capsule 112. For example, packaging, covering, seals, etc. may be removed from the capsule 112. In some embodiments, the capsule 112 may include at least one peel-off seal that covers over the openings in the first end 161 and the second end 163.

Then, the capsule 112 may be placed on and may be engaged with the base unit 110 (i.e., moved to an engaged position with the base unit 110 as shown, for example, in FIGS. 1, 4, and 5). Specifically, the capsule 112 may be centered with respect to the axis 106 and dropped into the receptacle 136. As shown in FIGS. 4 and 5, the upper rim 188 of the capsule 112 may rest on the inner ledge 140 when seated in the receptacle 136. Also, the taper dimension of the ribs 141 may substantially correspond to the taper of the outer wall 166 of the capsule 112 such that the outer wall 166 lies against and snugly nests against the ribs 141 on the side member 124 of the base unit 110. Also, the size and shape of the circular terminal end 168 of the capsule 112 may correspond to that of the outer ledge 145 of the base unit 110 such that the terminal end 168 snugly fits and nests on the outer ledge 145 of the base unit 110. Accordingly, the capsule 112 and the receptacle 136 may correspond in shape and size. Both the receptacle 136 and the housing 162 of the capsule 112 may be cup-shaped with rounded (e.g., circular) cross sections taken normal to the axis 106. Both the receptacle 136 and the capsule 112 may be aligned and centered on the axis 106 with corresponding widths (i.e., diameters) and tapered surfaces. As such, the capsule 112 may nest within the receptacle 136 and may be secured therein.

Furthermore, as shown in FIGS. 4 and 5, an airflow fluid coupling 149 may be established between the capsule 112 and the base unit 110 as a result of the capsule 112 engaging with the base unit 110. Specifically, the air outlet 150 of the base unit 110 may fluidly connect to the air inlet 176 of the capsule 112 when the capsule 112 is supported within the receptacle 136. Placement of the capsule 112 on the base unit 110 may coincidentally fluidly connect and align the air inlet 176 to the air outlet 150 of the base unit 110. In some embodiments, the air inlet 176 covers over an entirety of the air outlet 150 of the base unit 110. Stated differently, the air inlet 176 surrounds the base unit 110 with respect to the axis 106 (e.g., the air inlet 176 encircles the air outlet 150). Also, the terminal end 168 seats against the outer ledge 145 to block leakage flow between the outside of the capsule 112 and the base unit 110. In this position, the receptacle 136, the air outlet 150, the first end 161 of the capsule 112, the air inlet 176, the second end 163, and the outlet port 196 may be coaxial and centered with respect to the longitudinal axis 106. Also, in this position, the air outlet 150, and the air inlet 176 may be substantially aligned along the longitudinal axis 106.

Then, the fan 154 may be turned ON by the control system 158. For example, the user may push the input device 127, and the control system 158 may command the rotor 157 to begin rotating the rotor 157 of the fan 154 for a set time period. The fan 154 may draw air into the inlet apertures 132 and blow the air out of air outlet 150. The airflow 116 may be received and directed by the air inlet 176 and into the housing 162 of the capsule 112. The airflow 116 may be directed into the through-ways 202 of the volatile substance member 114. The airflow 116 may, therefore, pass through the member 114, into a so-called headspace 269 of the capsule 112 defined axially between the volatile substance member 114 and the cover member 192 of the capsule 112. The airflow 116 may eventually exit the capsule 112 via the apertures 194. As long as the rotor 157 of the fan 154 is powered ON, the airflow 116 may be continuously driven from the inlet apertures 132 of the base unit 110 and out of the capsule 112 via the apertures 194, and volatile material from the member 114 may be carried away into the surrounding air.

After the predetermined time period, the control system 158 may automatically turn the fan 154 OFF. If needed, the user may use the input device 127 to “manually” turn the fan 154 OFF, for example, by pressing the input device 127 multiple times (e.g., three times) in quick succession. The capsule 112 may remain in the receptacle 136 and engaged with the base unit 110 while the fan 154 is OFF. As such, the capsule 112 can remain in standby for when the fan 154 is again turn ON for delivering the volatiles.

Referring now to FIGS. 5 and 6, interior features of the volatile substance distribution system 100 will be discussed according to various embodiments of the present disclosure. The fan 154, various features of the housing 122, the chassis 151, as well as the capsule 112 may be arranged in a vertical stack 400 (FIG. 5) that is substantially aligned and centered on the axis 106. This arrangement is highly compact, provides stability, and is also convenient for use and for manufacturing purposes. This arrangement also provides ergonomic benefits to the user when placing the capsule 112 on the base unit 110 and when removing the capsule 112 from the base unit 110. Additionally, as will be discussed, the stack 400 defines an airflow system 402. In this airflow system 402, the housing 122 defines at least one fluid passage 403 extending from the inlet apertures 132, through the chassis 151, and to the air outlet 150 to provide the airflow 116 to the capsule 112. This airflow system 402 operates at high efficiency due to various features described herein. Because of this high-efficiency operation, the power consumption of the fan 154 may be relatively low. Thus, the fan 154 may be small and compact. Also, there may be relatively few batteries 155, and the batteries 155 that are included can be lightweight and arranged compactly.

The fan 154 may include particular features that benefit the airflow system 402. In some embodiments, the fan 154 may include four blades 404 that extend out radially from a hub 406 of the rotor 157. The hub 406 may have a diameter of approximately 0.5 inches in some embodiments. The outer radial edges of the blades 404 may collectively define an outer radial fan profile 408 (FIG. 6). The outer radial fan profile 408 may define an imaginary cylinder (e.g., a right circular cylinder) that is centered on the axis 106 and that extends parallel to the axis 106. The fan profile 408 may have a diameter of approximately two (2) inches in some embodiments. Furthermore, in some embodiments, the blades 404 may have a centerline that is tipped relative to the radial axes 108, 109 at an angle of attack, which may be approximately 29.68 degrees in some embodiments. Additionally, there may a gradual reduction in angle of attack at the tip of the blade 404 as compared to the hub 406 to define a linear twist, which may be approximately 9.68 degrees in some embodiments. Moreover, the blades 404 may have a chord length measured from the leading edge to the trailing edge, which may be approximately 0.362 inches at the hub 406, and which may gradually increase as the blade 404 extends further outward radially.

The fan 154 may be configured as a shrouded fan. In some embodiments, for example, the inner member 134 of the housing 122 may include a shroud member 410. The shroud member 410 may be tubular and hollow. The shroud member 410 may be fixedly attached to the side wall 142 of the inner member 134. In some embodiments, the shroud member 410 may be a thin-walled structure with an arcuate (e.g., semi-circular) cross section taken perpendicular to the axis 106. The shroud member 410 may be, in some embodiments, defined by a wall that extends almost continuously about the axis 106 in the circumferential direction; however, as shown in FIG. 4, this wall of the shroud member 410 may include an opening, notch, or other aperture 411 that interrupts the shroud member 410 in the circumferential direction. The aperture 411 may be disposed proximate user interface 125, the abutment member 302, and the components associated therewith.

The shroud member 410 may include a longitudinal segment 412 that is hollow and substantially tubular. The longitudinal segment 412 may depend from the lower end of the side wall 142. The longitudinal segment 412 may be centered on the axis 106. The longitudinal segment 412 may define an arcuate terminal end of the inner member 134 that is supported proximate the chassis 151. The longitudinal segment 412 may include an inner shroud surface 416. The inner shroud surface 416 may have an arcuate, semi-circular cross section. The inner shroud surface 416 may have a width (i.e., diameter) that remains substantially constant along its longitudinal length. Accordingly, the inner shroud surface 416 may substantially define a right circular cylinder in some embodiments. The shroud surface 416 may partly define the fluid passage 403 through the system 100, extending substantially along the axis 106 (e.g., parallel to the axis 106) and contouring about the axis 106 in the circumferential direction.

The shroud member 410 may further include a tapered segment 414. The tapered segment 414 may be frusto-conic and hollow. The tapered segment 414 may be connected at its lower end to the longitudinal segment 412, and the tapered segment 414 may project inward and longitudinally in the downstream direction therefrom. The lower support 144 of the receptacle 136 may be attached to the upper end of the tapered segment 414. As such, the tapered segment 414 may be disposed between the longitudinal segment 412 and the air outlet 150. The tapered segment 414 may include a tapered inner surface 418. The tapered inner surface 418 may have an arcuate (e.g., semi-circular) cross section taken perpendicular to the axis 106. The tapered inner surface 418 may have a width (i.e., diameter) that tapers and reduces gradually as it extends downstream along the axis 106.

The shroud member 410 may receive the fan 154. In some embodiments, the blades 404 may be received and surrounded in the circumferential direction by the longitudinal segment 412. The tapered segment 414 may be disposed slightly downstream of the fan 154 as shown in FIG. 5.

As shown in FIG. 6, the shroud surface 416 may radially oppose outer radial edges 420 of the blades 404. The shroud surface 416 and the fan profile 408 may have corresponding contour. For example, the shroud surface 416 and the fan profile 408 may both define right circular cylinders that are centered on the axis 106, wherein the fan profile 408 has a slightly smaller diameter than that of the shroud surface 416. Accordingly, a relatively small gap may be defined radially between the shroud surface 416 and the outer radial edges 420 of the blades 404. As such, operating efficiency of the fan 154 may be increased, backflow can be reduced, etc.

Furthermore, the tapered inner surface 418 of the shroud member 410 may direct and funnel the airflow 116 toward the capsule 112 in a controlled manner. The tapered inner surface 418 may focus the flow for effective delivery to the capsule 112.

Accordingly, the airflow system 402 may be highly efficient. The airflow system 402 may direct the airflow 116 efficiently from the base unit 110 to the capsule 112. The airflow system 402 may also operate at low noise levels. Furthermore, the system 100 may be very compact and highly ergonomic. In addition, manufacture of the base unit 110 may be relatively efficient because there are relatively few parts and because assembly is relatively simple.

Terms such as “first” and “second” have been utilized above to describe similar features or characteristics (e.g., longitudinal directions) in view of the order of introduction during the course of description. In other sections of this Application, such terms can be varied, as appropriate, to reflect a different order of introduction. While at least one exemplary embodiment has been presented in the foregoing Detailed Description, it should be appreciated that a vast number of variations exist. It should also be appreciated that the exemplary embodiment or exemplary embodiments are only examples, and are not intended to limit the scope, applicability, or configuration of the present disclosure in any way. Rather, the foregoing Detailed Description will provide those skilled in the art with a convenient road map for implementing an exemplary embodiment of the present disclosure. It is understood that various changes may be made in the function and arrangement of elements described in an exemplary embodiment without departing from the scope of the present disclosure as set forth in the appended claims.

What is claimed is:

1. A volatile substance distribution system comprising:
a fan having a fan profile;
a housing that defines a fluid passage, the fan disposed in the fluid passage, the housing defining a receptacle configured to removably receive a capsule containing a volatile substance member, the fluid passage fluidly connected to the capsule when the receptacle removably receives the capsule; and
a shroud surface that partly defines the fluid passage and that opposes the fan, the shroud surface and the fan profile having a corresponding contour.

2. The volatile substance distribution system of claim 1, wherein the fan is supported for rotation about an axis; and
wherein the shroud surface extends substantially along the axis and contours about the axis in a circumferential direction.

3. The volatile substance distribution system of claim 2, wherein the shroud surface extends substantially parallel to the axis and contours about the axis in a circumferential direction.

4. The volatile substance distribution system of claim 2, wherein the housing includes an aperture that fluidly connects with the capsule when removably received in the receptacle;
further comprising a tapered surface that partly defines the fluid passage and that has a tapering width with respect to the axis, the tapered surface disposed axially between the shroud surface and the aperture.

5. The volatile substance distribution system of claim 4, wherein the aperture is an outlet aperture of the fluid passage from the housing.

6. The volatile substance distribution system of claim 5, wherein the tapered surface has an arcuate cross section taken perpendicular to the axis;
wherein the tapering width reduces as the tapered surface extends from the shroud surface toward the aperture.

7. The volatile substance distribution system of claim 1, wherein the fan is supported for rotation about an axis;
wherein the receptacle and the shroud surface are substantially coaxial and substantially centered on the axis.

8. The volatile substance distribution system of claim 7, further comprising a chassis that supports the fan; and
wherein the chassis includes at least one aperture that is substantially centered on the axis.

9. The volatile substance distribution system of claim 8, wherein the aperture defines an equilateral triangular profile.

10. The volatile substance distribution system of claim 9, wherein the chassis supports a plurality of batteries that are arranged end-to-end in an equilateral triangular formation that is centered on the axis.

11. The volatile substance distribution system of claim 1, wherein the housing includes an inner member that defines the receptacle and that includes a shroud member that is attached to the inner member and that defines the shroud surface.

12. The volatile substance distribution system of claim 11, wherein the inner member includes a side wall and a lower support that cooperatively define the receptacle; and
wherein at least part of the shroud member depends from the side wall.

13. The volatile substance distribution system of claim 12, further comprising a chassis that supports the fan; and
wherein at least part of the shroud member depends from the side wall and terminates proximate the chassis.

14. The volatile substance distribution system of claim 1, comprising the capsule that is removably received in the receptacle;
wherein the fan is supported for rotation about an axis;
wherein the capsule and the shroud surface are substantially centered on the axis.

15. A method of manufacturing a volatile substance distribution system comprising:
supporting a fan within a fluid passage of a housing, the fan having a fan profile, the housing defining a receptacle configured to removably receive a capsule containing a volatile substance member, the fluid passage fluidly connected to the capsule when the receptacle removably receives the capsule; and
supporting the fan within a shroud surface that partly defines the fluid passage and that opposes the fan, the shroud surface and the fan profile having a corresponding contour.

16. The method of claim 15, further comprising supporting the fan for rotation about an axis; and
wherein the shroud surface extends substantially along the axis and contours about the axis in a circumferential direction.

17. The method of claim 16, wherein the shroud surface extends substantially parallel to the axis and contours about the axis in a circumferential direction.

18. The method of claim 16, wherein the housing includes an aperture that fluidly connects with the capsule when removably received in the receptacle;
further comprising a tapered surface that partly defines the fluid passage and that has a tapering width with respect to the axis, the tapered surface disposed axially between the shroud surface and the aperture.

19. The method of claim 18, wherein the tapered surface has an arcuate cross section taken perpendicular to the axis;
wherein the tapering width reduces as the tapered surface extends from the shroud surface toward the aperture.

20. A volatile substance distribution system comprising:
a capsule containing a volatile substance member; and
a base unit with a housing that houses a fan, the fan having a fan profile, the housing defining a receptacle that removably receives the capsule, the housing defining a fluid passage that fluidly connects to the capsule, the fan disposed in the fluid passage, the fluid passage partly defined by a shroud surface of the housing, the shroud surface opposing the fan, the shroud surface and the fan profile having a corresponding contour.
